(19) United States
(12) Patent Application Publication
(10) Pub. No.: US 2007/0276174 A1
(43) Pub. Date: Nov. 29, 2007

(54) Synthesis of molecular sieves and their use in the conversion of oxygenates to olefins

(76) Inventors: Luc R.M. Martens, Meise (BE); Machteld Maria Mertens, Boortmeerbeek (BE); Goetz Burgfels, Bad Aibling (DE); Marcus Breuninger, Rosenheim (DE); Andreas Pritzl, Bad Aibling (DE)

Correspondence Address: ExxonMobil Chemical Company;Law Technology, P.O. Box 2149, Baytown, TX, 77522-2149, US

(21) Appl. No.: 11/784,364

(22) Filed: Apr. 6, 2007

Related U.S. Application Data
(60) Provisional application No. 60/809,101, filed on May 26, 2006.

(51) Int. Cl. C07C 1/20; B01J 27/14; B01J 27/186; B01J 33/00

(52) U.S. Cl. 585/639; 422/40; 502/208; 502/214

(57) ABSTRACT

In a method of synthesizing a crystalline molecular sieve, a reaction mixture is formed comprising a source of phosphorus, a source of aluminum, at least one organic directing agent and, optionally, a source of silicon and crystallization of the reaction mixture is induced to form a slurry comprising the desired crystalline molecular sieve. The slurry is then maintained in contact with a flocculant for a period of 12 hours to 30 days before the crystalline molecular sieve is recovered from said slurry.

### CROSS REFERENCE TO RELATED APPLICATIONS

This claims the benefit of and priority from U.S. Ser. No. 60/809,101, filed May 26, 2006. The above application is fully incorporated herein by reference.

### FIELD OF THE INVENTION

This invention relates to a method of synthesizing crystalline molecular sieves and, in particular, silicoaluminophosphate and aluminophosphate molecular sieves.

### BACKGROUND OF THE INVENTION

Silicoaluminophosphate (SAPO) and aluminophosphate (ALPO) molecular sieves are well known and have been used in a variety of applications, for example, as adsorbents and catalysts. As catalysts, SAPOs and ALPOs have been used in processes such as fluid catalytic cracking, hydrocracking, isomerization, oligomerization, the conversion of alcohols or ethers, and the alkylation of aromatics. In particular, considerable interest has been focused on the use of SAPOs and ALPOs in converting alcohols or ethers to olefin products, particularly ethylene and propylene. Since the commercial application of such a process will require very large catalyst loadings, typically in the range of from 1,000 kg to 700,000 kg, considerable research has also been directed to improving the synthesis of SAPOs and ALPOs.

Generally, the synthesis of silicoaluminophosphate and aluminophosphate molecular sieves involves preparing a mixture comprising a source of water, a reactive source of aluminum, a reactive source of phosphorus, at least one organic directing agent for directing the formation of said molecular sieve and, optionally, a reactive source of silicon. The reaction mixture is then heated, normally with agitation, to a suitable crystallization temperature, typically between about 100 and about 300° C., and then held at this temperature for a sufficient time, typically between about 1 hour and 20 days, for crystallization of the desired molecular sieve to occur. The molecular sieve crystals are then separated from the reaction mixture, for example, by centrifugation or filtration; washed, typically with deionized water; and then dried before being subjected to activation and catalyst particle formation.

In the larger scale syntheses required for the production of quantities of molecular sieve necessary for pilot plant scale-up or commercial production, there is frequently a delay between the termination of the crystallization and the separation and washing of the crystals from the reaction mixture. It has, however, been found that such delays, in which the as-synthesized crystals are still in contact with the reaction mixture, can result in redissolution of the crystals into the reaction mixture and hence in loss of yield of the molecular sieve product. In fact, laboratory scale tests suggest that there can be a loss in the yield of SAPO molecular sieve of 2% or more if the as-synthesized crystals are allowed to remain in contact with the reaction mixture, without washing, for two days.

According to the invention, it has now been found that the problem of the SAPO and ALPO crystals redissolving on prolonged contact with the reaction mixture can be alleviated by the presence of a flocculant during such contact.

U.S. Pat. No. 7,014,827 relates to a process for the synthesis and recovery of silicoaluminophosphate (SAPO) and/or aluminophosphate (ALPO) molecular sieves and also seeks to alleviate the problem of loss of crystalline material during storage. In particular, the application discloses a process for synthesizing a crystalline molecular sieve by: (a) forming a reaction mixture comprising a source of alumina, a source of phosphate, at least one nitrogen-containing organic directing agent, and optionally a source of silica; (b) inducing crystallization of the crystalline molecular sieve from the reaction mixture to form a slurry comprising the crystalline molecular sieve; and (c) recovering the crystalline molecular sieve from the slurry, wherein during any period of time after substantial completion of the crystallization in step (b), and prior to the recovery step (c), the slurry is held under substantially static conditions.

U.S. Pat. No. 5,296,208 discloses a process for synthesizing a crystalline microporous molecular sieve which comprises the steps of: (a) forming an aqueous reaction mixture suitable for the hydrothermal production of a crystalline molecular sieve, said reaction mixture containing at least one nitrogen-containing organic directing agent; (b) establishing a crystallization period by heating and maintaining said reaction mixture at a crystallization temperature of at least 100° C. to form crystals of the molecular sieve product; and (c) recovering the crystallized product from the reaction mixture. In particular, the reaction mixture formed in step (a) is arranged to contain organic base in excess of the amount to be incorporated within the product molecular sieve crystals, and thereafter at least some of said excess organic base is removed during the course of the crystallization period of step (b) whereby the equilibrium between molecular sieve product formation and molecular sieve product dissolution of the reaction system is shifted in favor of decreased dissolution of molecular sieve product at the existing temperature conditions. According to column 7, line 58 to column 8, line 2, the removal of organic base as a means of decreasing the dissolution of crystalline molecular sieve product can be augmented by other techniques, for example, by rapidly decreasing the concentration of organic base in the reaction mixture by dilution with water, by rapidly decreasing the temperature of the reaction system; or by a combination thereof, such as by diluting with chilled water.

U.S. Pat. No. 7,122,500 discloses a method for synthesizing a molecular sieve, such as an aluminophosphate or a silicoaluminophosphate, the method comprising the steps of: (a) crystallizing the molecular sieve in a slurry, the slurry comprising one or more of a silicon source, an aluminum source, and a phosphorus source; (b) contacting a flocculant with the molecular sieve; (c) recovering the molecular sieve; and (d) heat treating the molecular sieve. The flocculant is used to increase the recovery rate of the molecular sieve crystals and hence increase the yield of the synthesized molecular sieve crystals. There is no disclosure or suggestion of the flocculant retarding redissolution of the molecular sieve crystals.

U.S. Patent Application Publication No. 2005-0256354 discloses a process for producing one or more olefin(s), the process comprising the steps of: (a) introducing a feedstock to a reactor system in the presence of a molecular sieve catalyst composition comprising a synthesized molecular sieve having been recovered in the presence of a flocculant; (b) withdrawing from the reactor system an effluent stream; and (c) passing the effluent gas through a recovery system recovering at least the one or more olefin(s). Again, the flocculant is used to increase the recovery rate of the molecular sieve crystals and there is no disclosure or suggestion of the flocculants being effective to retard redissolution of the molecular sieve crystals. According to paragraph [0037], the flocculated sieve can be recovered from the synthesis mixture by centrifugation, filtration or by allowing the mixture to settle, decanting the liquid, re-slurrying with water, then repeatedly decanting and re-slurrying, and finally recovering by centrifugation or filtration. The settling of the sieve can take from seconds to days; however, the settling can be accelerated by adding additional flocculant.

### SUMMARY OF THE INVENTION

In one aspect, the present invention resides in a method of synthesizing a crystalline molecular sieve, the method comprising:

(a) forming a reaction mixture comprising a source of phosphorus, a source of aluminum, at least one organic directing agent, and optionally a source of silicon;

(b) inducing crystallization of the crystalline molecular sieve from said reaction mixture to form a slurry, the slurry comprising said crystalline molecular sieve;

(c) maintaining said slurry in contact with a flocculant for a period of 12 hours to 30 days; and thereafter

(d) recovering said crystalline molecular sieve from said slurry.

Conveniently, said slurry is maintained in contact with said flocculant for a period of 12 hours to 30 days, for example, for a period of 24 hours to 20 days, such as for a period of 48 hours to 10 days, such as for a period of 72 hours to 5 days.

Conveniently, the amount of flocculant present during (c) is between about 0.005% and about 0.100%, preferably between about 0.01% and about 0.05%, by weight of the crystalline molecular sieve.

In one embodiment, the method comprises the further step of diluting the slurry with water so that the volume ratio of slurry to water diluent is between 1:0.5 and 1:1.5, preferably between 1:0.7 and 1:1.2.

Preferably, said flocculant is an organic polymer, such as a polyethyleneimine.

In one embodiment, said crystalline molecular sieve is selected from a CHA framework-type molecular sieve, an AEI framework-type molecular sieve, and an intergrowth of CHA and AEI framework-type molecular sieves.

In a further aspect, the invention resides in a method of increasing the storage life of an as-synthesized silicoaluminophosphate and/or an aluminophosphate molecular sieve comprising adding a flocculant to a slurry comprising crystals of said molecular sieve and a liquid medium used in the crystallization of said molecular sieve.

In yet a further aspect, the invention resides in the use of a flocculant to inhibit redissolution of an as-synthesized silicoaluminophosphate and/or an aluminophosphate molecular sieve in a liquid medium used in the crystallization of said molecular sieve.

In still yet a further aspect, the invention resides in a molecular sieve synthesized by a method described herein and its use in the conversion of an oxygenate-containing feedstock to a product comprising olefins.

### BRIEF DESCRIPTION OF THE DRAWINGS

FIG. 1 is a graph plotting silicoaluminophosphate yield against time of storage for the as-synthesized slurries of Example 1 (untreated slurry), Example 2 (slurry diluted with an equal weight of deionized water) and Example 3 (slurry diluted with an equal weight of deionized water and mixed with a flocculant such that weight ratio of flocculant to slurry was 0.8:1).

FIG. 2 is a scanning electron micrograph of the washed as-synthesized slurry of Example 1 after storage for 60 hours.

FIG. 3 is a scanning electron micrograph of the untreated as-synthesized slurry of Example 1 after storage for 19 days.

FIG. 4 is a scanning electron micrograph of the slurry of Example 2 after storage for 19 days.

FIG. 5 is a scanning electron micrograph of the slurry of Example 3 after storage for 19 days.

### DETAILED DESCRIPTION OF THE INVENTION

The present invention provides a method of synthesizing crystalline molecular sieves, particularly phosphorus-containing crystalline molecular sieves, such as silicoaluminophosphates and/or aluminophosphates, in which the slurry containing the as-synthesized molecular sieve crystals is stored in the presence of a flocculant, preferably a polymeric organic flocculant. In this way, it is found that the tendency of the as-synthesized crystals to redissolve in the slurry is significantly reduced, thereby enhancing the overall yield of molecular sieve resulting from the synthesis process.

### Molecular Sieves

Crystalline molecular sieves have a three-dimensional, four-connected framework structure of corner-sharing [TO₄] tetrahedra, where T is any tetrahedrally coordinated cation. The molecular sieves produced by the present method are conveniently silicoaluminophosphates (SAPOs), in which the framework structure is composed of [SiO₄], [AlO₄] and [PO₄] corner sharing tetrahedral units, or aluminophosphates (ALPOs), in which the framework structure is composed of [AlO₄] and [PO₄] corner sharing tetrahedral units.

Molecular sieves have been classified by the Structure Commission of the International Zeolite Association according to the rules of the IUPAC Commission on Zeolite Nomenclature. According to this classification, framework-type zeolite and zeolite-type molecular sieves, for which a structure has been established, are assigned a three letter code and are described in the Atlas of Zeolite Framework Types, 5th edition, Elsevier, London, England (2001), which is fully incorporated herein by reference.

Non-limiting examples of the molecular sieves for which a structure has been established include the small pore molecular sieves of a framework type selected from the group consisting of AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof; the medium pore molecular sieves of a framework type selected from the group consisting of AFO, AEL, EUO, HEU, FER, MEL, MFI, MTW, MTT, TON, and substituted forms thereof; and the large pore molecular sieves of a framework-type selected from the group consisting of EMT, FAU, and substituted forms thereof. Other molecular sieves have a framework type selected from the group consisting of ANA, BEA, CFI, CLO, DON, GIS, LTL, MER, MOR, MWW, and SOD.

Non-limiting examples of the preferred molecular sieves, particularly for converting an oxygenate containing feedstock into olefin(s), include those having a framework-type selected from the group consisting of AEL, AFY, BEA, CHA, EDI, FAU, FER, GIS, LTA, LTL, MER, MFI, MOR, MTT, MWW, TAM, and TON.

Molecular sieves are typically described in terms of the size of the ring that define a pore, where the size is based on the number of T atoms in the ring. Small pore molecular sieves generally have up to 8-ring structures and an average pore size less than 5 Å, whereas medium pore molecular sieves generally have 10-ring structures and an average pore size of about 5 Å to about 6 Å. Large pore molecular sieves generally have at least 12-ring structures and an average pore size greater than about 6 Å. Other framework-type characteristics include the arrangement of rings that form a cage, and when present, the dimension of channels, and the spaces between the cages. See van Bekkum, et al., Introduction to Zeolite Science and Practice, Second Completely Revised and Expanded Edition, Vol. 137, pp. 1-67, Elsevier Science, B.V., Amsterdam, Netherlands (2001).

Conveniently, the silicoaluminophosphate and aluminophosphate molecular sieves produced by the method of the invention are small pore materials having an AEI topology or a CHA topology, such as SAPO-18 or SAPO-34, or including at least one intergrowth of an AEI framework-type material and a CHA framework-type material. Examples of such intergrowth materials are described in International Patent Publication Nos. WO 98/15496 and WO 02/70407, the entire disclosures of which are fully incorporated herein by reference.

### Molecular Sieve Synthesis

Generally, molecular sieves are synthesized by the hydrothermal crystallization of one or more of a source of aluminum, a source of phosphorus, a source of silicon, an organic directing agent, and a metal containing compound. Typically, a combination of sources of silicon, aluminum and phosphorus, optionally, with one or more organic directing agents and/or one or more metal-containing compounds, are dissolved or slurried in water and are placed in a sealed pressure vessel, optionally, lined with an inert plastic such as polytetrafluoroethylene, and heated under pressure at static or stirred conditions until a crystalline material is formed in a synthesis mixture. Typically crystallization is conducted at a temperature between about 100 and about 300° C. for a time between about 1 hour and 20 days.

When crystallization is complete, the liquid portion of the synthesis mixture is removed, decanted, or reduced in quantity to allow recovery of the crystalline molecular sieve. In a commercial process, one or more flocculant(s) may be added to the synthesis mixture to assist in the recovery of the molecular sieve crystals by promoting agglomeration of very small particles into larger aggregates of molecular sieve crystals. The crystalline molecular sieve is then separated from the synthesis mixture, typically by centrifugation or filtration; and then washed, typically with deionized water, to remove any residual synthesis mixture. After washing, the crystalline material is dried before being subjected to activation and catalyst particle formation.

In a large-scale commercial process, production schedules may require that there is a delay between the termination of the crystallization and the separation and washing of the crystals from the synthesis mixture. It has, however, been found that such delays, in which the as-synthesized crystals are stored in contact with the synthesis mixture, can result in redissolution of the crystals into the synthesis mixture and hence in loss of yield of the molecular sieve product. According to the invention, it has now been found that the problem of the molecular sieves crystals redissolving on prolonged contact with the synthesis mixture can be alleviated by adding a flocculant to the unwashed molecular sieve crystals.

Typically, the as-synthesized crystals may be stored in contact with the synthesis mixture for a period of 12 hours to 30 days, such as for a period of 24 hours to 20 days, for example, for a period of 48 hours to 10 days, such as for a period of 72 hours to 5 days. In the absence of a flocculant, yield losses in excess of 2% have been encountered after only two days storage, with essentially complete redissolution of the crystals after 20 days. However, by maintaining the as-synthesized crystals in contact with a suitable flocculant during such a storage period, it has been found that redissolution of the crystals into the synthesis mixture can be reduced to less than 1.5% even with storage times of 20 days.

There are many types of flocculants, including both inorganic and organic flocculants, suitable for use in the method of the invention. Inorganic flocculants are typically aluminum or iron salts that form insoluble hydroxide precipitates in water. Non-limiting examples include aluminum sulfate, poly(aluminum chloride), sodium aluminate, iron(III)-chloride, sulfate, and sulfate-chloride, iron(II)sulfate, and sodium silicate (activated silica). The major classes of flocculants are: (1) nonionic flocculants, for example, polyethylene oxide, polyacrylamide (PAM), partially hydrolyzed polyacrylamide (HPAM), and dextran; (2) cationic flocculants, for example, polyethyleneimine (PEI), polyacrylamide-co-trimethylammonium, ethyl methyl acrylate chloride (PTAMC), and poly(N-methyl-4-vinylpyridinium iodide); and (3) anionic flocculants, for example, poly(sodium acrylate), dextran sulfates, alum (aluminum sulfate), and/or high molecular weight ligninsulfonates prepared by a condensation reaction of formaldehyde with ligninsulfonates, and polyacrylamide. In a preferred embodiment, where the synthesis mixture includes the presence of water, it is preferable that the flocculant used is water soluble. Additional information on flocculation is discussed in T. C. Patton, Paint Flow and Pigment Dispersion—A Rheological Approach to Coating and Ink Technology, 2nd Edition, John Wiley & Sons, New York, p. 270, 1979, which is fully incorporated herein by reference.

Conveniently, the flocculant is added to the as-synthesized crystals or the synthesis mixture after crystallization in an amount of about 0.005 to about 0.100 wt %, preferably from about 0.01 to about 0.05 wt %, more preferably from about 0.15 to about 0.04 wt % flocculant based on the solid molecular sieve product. The flocculant is typically added to the slurry at room temperature, and is preferably added as a solution. If a solid flocculant is used then it is preferable that a substantially homogeneous flocculant solution is prepared by dissolving the solid flocculant in a liquid medium, preferably water. In a preferred embodiment, the synthesis mixture is diluted with water, preferably deionized water, in addition to the flocculant treatment so that the weight ratio of slurry to water diluent is between 1:0.5 and 1:1.5, preferably between 1:0.7 and 1:1.2. The dilution further aids in inhibiting redissolution of the as-synthesized molecular sieve crystals.

### Production of Molecular Sieve Catalyst Composition

As a result of the crystallization process, the recovered crystalline molecular sieve typically contains within its pores at least a portion of the organic directing agent used in the synthesis. Thus production of a catalyst composition from the as-synthesized molecular sieve generally involves an activation step, in which the organic directing agent is removed from the molecular sieve, leaving active catalytic sites within the microporous channels of the molecular sieve open for contact with a feedstock. The activation process is typically accomplished by calcining, or essentially heating the molecular sieve comprising the template at a temperature of from about 200° C. to about 800° C. in the presence of an oxygen-containing gas. In some cases, it may be desirable to heat the molecular sieve in an environment having a low or zero oxygen concentration. This type of process can be used for partial or complete removal of the organic directing agent from the intracrystalline pore system. In other cases, particularly with smaller organic directing agents, complete or partial removal from the sieve can be accomplished by conventional desorption processes.

In addition to activation, catalyst formulation normally includes combining the molecular sieve with other materials, such as binders and/or matrix materials, which provide additional hardness or catalytic activity to the finished catalyst. Such materials can be inert or catalytically active and include compositions such as kaolin and other clays, various forms of rare earth metals, other non-zeolite catalyst components, zeolite catalyst components, alumina or alumina sol, titania, zirconia, quartz, silica or silica sol, and mixtures thereof. These components are also effective in reducing overall catalyst cost, acting as a thermal sink to assist in heat shielding the catalyst during regeneration, densifying the catalyst and increasing catalyst strength. When blended with such components, the amount of molecular sieve contained in the final catalyst product ranges from 10 to 90 weight percent of the total catalyst, preferably 20 to 70 weight percent of the total catalyst.

### Uses of the Molecular Sieve

The crystalline molecular sieve produced by the method of the invention can be used to dry gases and liquids; for selective molecular separation based on size and polar properties; as an ion-exchanger; as a chemical carrier; in gas chromatography; and as a catalyst in organic conversion reactions. Examples of suitable catalytic uses of the crystalline material produced by the method of the invention include: (a) hydrocracking of heavy petroleum residual feedstocks, cyclic stocks and other hydrocrackate charge stocks, normally in the presence of a hydrogenation component is elected from Groups 6 and 8 to 10 of the Periodic Table of Elements; (b) dewaxing, including isomerization dewaxing, to selectively remove straight chain paraffins from hydrocarbon feedstocks typically boiling above 177° C., including raffinates and lubricating oil basestocks; (c) catalytic cracking of hydrocarbon feedstocks, such as naphthas, gas oils and residual oils, normally in the presence of a large pore cracking catalyst, such as zeolite Y; (d) oligomerization of straight and branched chain olefins having from about 2 to 21, preferably 2 to 5 carbon atoms, to produce medium to heavy olefins which are useful for both fuels, i.e., gasoline or a gasoline blending stock, and chemicals; (e) isomerization of olefins, particularly olefins having 4 to 6 carbon atoms, and especially normal butene to produce iso-olefins; (f) upgrading of lower alkanes, such as methane, to higher hydrocarbons, such as ethylene and benzene; (g) disproportionation of alkylaromatic hydrocarbons, such as toluene, to produce dialkylaromatic hydrocarbons, such as xylenes; (h) alkylation of aromatic hydrocarbons, such as benzene, with olefins, such as ethylene and propylene, to produce ethylbenzene and cumene; (i) isomerization of dialkylaromatic hydrocarbons, such as xylenes; (j) catalytic reduction of nitrogen oxides; and (k) synthesis of monoalkylamines and dialkylamines.

In particular, the crystalline material produced by the method of the invention is useful in the catalytic conversion of oxygenates to one or more olefins, particularly ethylene and propylene. As used herein, the term “oxygenates” is defined to include, but is not necessarily limited to aliphatic alcohols, ethers, carbonyl compounds (aldehydes, ketones, carboxylic acids, carbonates, and the like), and also compounds containing hetero-atoms, such as, halides, mercaptans, sulfides, amines, and mixtures thereof. The aliphatic moiety will normally contain from about 1 to about 10 carbon atoms, such as from about 1 to about 4 carbon atoms.

Representative oxygenates include lower straight chain or branched aliphatic alcohols, their unsaturated counterparts, and their nitrogen, halogen and sulfur analogues. Examples of suitable oxygenate compounds include methanol; ethanol; n-propanol; isopropanol; C₄-C₁₀ alcohols; methyl ethyl ether; dimethyl ether; diethyl ether; di-isopropyl ether; methyl mercaptan; methyl sulfide; methyl amine; ethyl mercaptan; di-ethyl sulfide; di-ethyl amine; ethyl chloride; formaldehyde; di-methyl carbonate; di-methyl ketone; acetic acid; n-alkyl amines, n-alkyl halides, n-alkyl sulfides having n-alkyl groups of comprising the range of from about 3 to about 10 carbon atoms; and mixtures thereof. Particularly suitable oxygenate compounds are methanol, dimethyl ether, or mixtures thereof, most preferably methanol. As used herein, the term “oxygenate” designates only the organic material used as the feed. The total charge of feed to the reaction zone may contain additional compounds, such as diluents.

In the present oxygenate conversion process, a feedstock comprising an organic oxygenate, optionally, with one or more diluents, is contacted in the vapor phase in a reaction zone with a catalyst comprising the molecular sieve produced by the method of the invention at effective process conditions so as to produce the desired olefins. Alternatively, the process may be carried out in a liquid or a mixed vapor/liquid phase. When the process is carried out in the liquid phase or a mixed vapor/liquid phase, different conversion rates and selectivities of feedstock-to-product may result depending upon the catalyst and the reaction conditions.

When present, the diluent(s) is generally non-reactive to the feedstock or molecular sieve catalyst composition and is typically used to reduce the concentration of the oxygenate in the feedstock. Non-limiting examples of suitable diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. Diluent(s) may comprise from about 1 mol % to about 99 mol % of the total feed mixture.

The temperature employed in the oxygenate conversion process may vary over a wide range, such as from about 200° C. to about 1000° C., for example, from about 250° C. to about 800° C., including from about 250° C. to about 750° C., conveniently from about 300° C. to about 650° C., typically from about 350° C. to about 600° C. and particularly from about 400° C. to about 600° C.

Light olefin products will form, although not necessarily in optimum amounts, at a wide range of pressures, including but not limited to autogenous pressures and pressures in the range of from about 0.1 kPa to about 10 MPa. Conveniently, the pressure is in the range of from about 7 kPa to about 5 MPa, such as in the range of from about 50 kPa to about 1 MPa. The foregoing pressures are exclusive of diluent, if any is present, and refer to the partial pressure of the feedstock as it relates to oxygenate compounds and/or mixtures thereof. Lower and upper extremes of pressure may adversely affect selectivity, conversion, coking rate, and/or reaction rate; however, light olefins such as ethylene still may form.

The process should be continued for a period of time sufficient to produce the desired olefin products. The reaction time may vary from tenths of seconds to a number of hours. The reaction time is largely determined by the reaction temperature, the pressure, the catalyst selected, the weight hourly space velocity, the phase (liquid or vapor) and the selected process design characteristics.

A wide range of weight hourly space velocities (WHSV) for the feedstock will function in the present process. WHSV is defined as weight of feed (excluding diluent) per hour per weight of a total reaction volume of molecular sieve catalyst (excluding inerts and/or fillers). The WHSV generally should be in the range of from about 0.01 hr⁻¹ to about 500 hr⁻¹, such as in the range of from about 0.5 hr⁻¹ to about 300 hr⁻¹, for example, in the range of from about 0.1 hr⁻¹ to about 200 hr⁻¹.

A practical embodiment of a reactor system for the oxygenate conversion process is a circulating fluid bed reactor with continuous regeneration, similar to a modern fluid catalytic cracker. Fixed beds are generally not preferred for the process because oxygenate to olefin conversion is a highly exothermic process which requires several stages with intercoolers or other cooling devices. The reaction also results in a high pressure drop due to the production of low pressure, low density gas.

Because the catalyst must be regenerated frequently, the reactor should allow easy removal of a portion of the catalyst to a regenerator, where the catalyst is subjected to a regeneration medium, such as a gas comprising oxygen, for example, air, to burn off coke from the catalyst, which restores the catalyst activity. The conditions of temperature, oxygen partial pressure, and residence time in the regenerator should be selected to achieve a coke content on regenerated catalyst of less than about 0.5 wt %. At least a portion of the regenerated catalyst should be returned to the reactor.

In one embodiment, the catalyst is pretreated with dimethyl ether, a C₂-C₄ aldehyde composition and/or a C₄-C₇ olefin composition to form an integrated hydrocarbon co-catalyst within the porous framework of the molecular sieve prior to the catalyst being used to convert oxygenate to olefins. Desirably, the pretreatment is conducted at a temperature of at least 10° C., such as at least 25° C., for example, at least 50° C., higher than the temperature used for the oxygenate reaction zone and is arranged to produce at least 0.1 wt %, such as at least 1 wt %, for example, at least about 5 wt % of the integrated hydrocarbon co-catalyst, based on total weight of the molecular sieve. Such preliminary treating to increase the carbon content of the molecular sieve is known as “pre-pooling” and is further described in U.S. Pat. Nos. 7,045,672; 7,057,083; and 7,132,581; and are fully incorporated herein by reference.

The invention will now be more particularly described with reference to the following Examples and the accompanying drawings.

### EXAMPLE 1 (COMPARATIVE)

An EMM-2 molecular sieve was synthesized by the following procedure. A mixture with the following molar composition:
0.12 SiO₂/Al₂O₃/P₂O₅/TEAOH/35H₂O
   was prepared by combining the following ingredients in the appropriate amounts: tetraethylammonium hydroxide, TEAOH [35% in water] and phosphoric acid [85% in water], Ludox AS40, Pural SB1, and water. This mixture was then crystallized by heating to 165° C. while agitating for 100 hrs.

After crystallization, the slurry was cooled to room temperature and 8 samples of the untreated slurry were sealed in separate polyethylene sample bottles and stored at room temperature for 1, 2, 5, 8, 12, 15, 19, and 22 days respectively.

At the end of its prescribed storage time, each bottle was opened and the molecular sieves crystals were immediately separated from the slurry by washing, filtration, and drying at 120° C. for 16 hours. The weight of the separated crystals was measured and the product yield (as a percentage of the total weight of the synthesis mixture) was plotted against storage time. The results are shown in FIG. 1, from which it will be seen that after 22 days storage the product yield had decreased from an initial value of 21.8 wt % to 1.6 wt %, indicating almost complete digestion of the molecular sieve crystals.

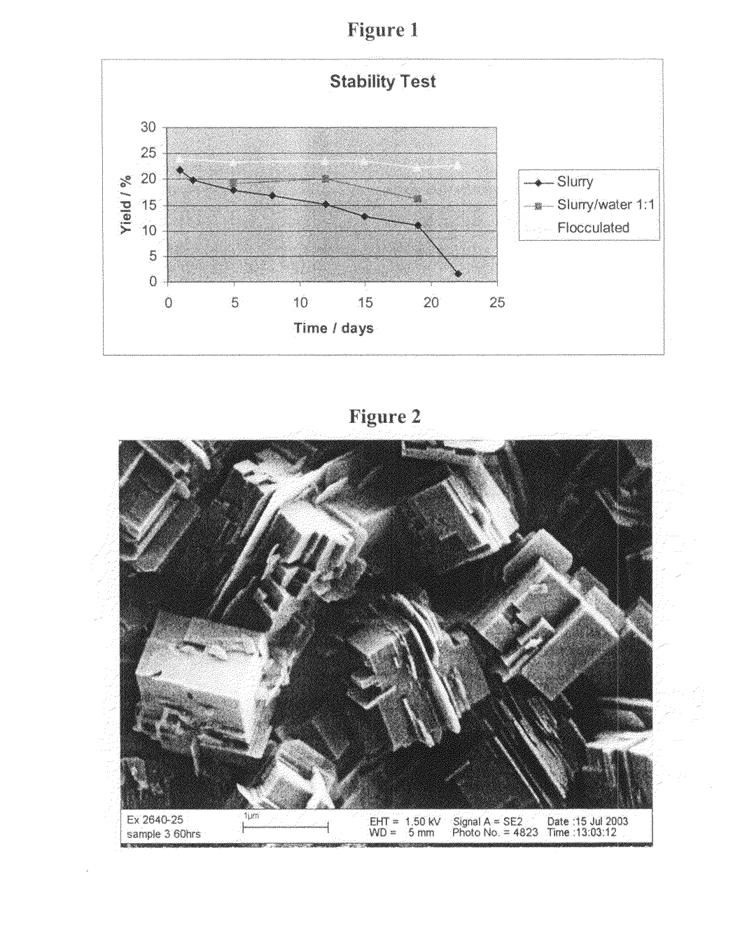

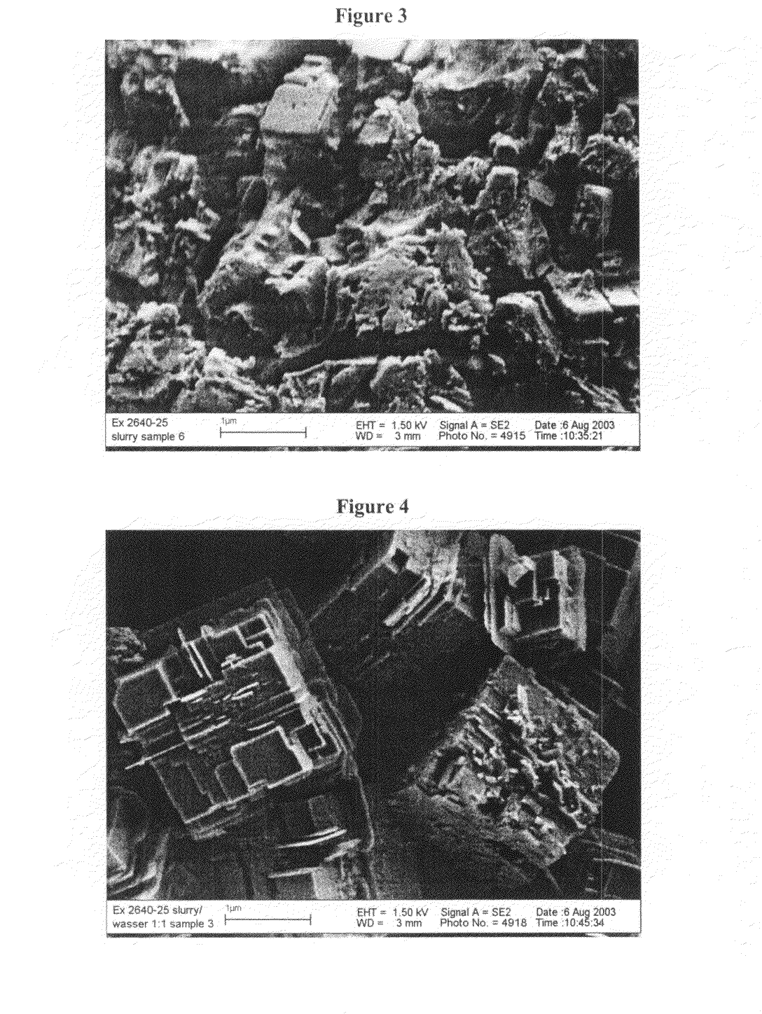

Scanning electron microscopy (SEM) of the untreated slurry after storage for 19 days also demonstrated almost complete dissolution of the molecular sieve crystals, starting with the formation of macropores in the morphology of the half cube crystals (see FIG. 3). By way of comparison, FIG. 2 is an SEM of a further sample of the untreated as-synthesized slurry, but which had been washed with deionized water immediately after crystallization was complete and which had been stored for 60 hours.

### EXAMPLE 2 (COMPARATIVE)

A further portion of the untreated slurry from the crystallization procedure described in Example 1 was diluted with an equal weight of deionized water and divided into 3 samples. Each sample was weighed into a polyethylene sample bottle, mixed for 3 minutes and subsequently sealed. The samples were then stored at room temperature for 5, 12, and 19 days respectively.

At the end of the storage time, each bottle was opened and the molecular sieve crystals were immediately separated from the slurry by washing, filtration and drying at 120° C. for 16 hours. The weight of the separated crystals was measured and the product yield (as a percentage of the total weight of the synthesis mixture) was plotted against storage time. The results are shown in FIG. 1, from which it will be seen that the diluted slurry was more stable than the undiluted slurry of Example 1, but significant dissolution of the molecular sieve crystals was apparent after 19 days storage. Dissolution was also evident in the SEM taken after 19 days storage (FIG. 4).

### EXAMPLE 3

A further portion of the untreated slurry from the crystallization procedure described in Example 1 was diluted with an equal weight of deionized water under stirring and a solution of a cationic polymer flocculant was added to the diluted slurry under slow agitation until flocks started to precipitate. The amount of flocculant added was such that the weight ratio of flocculant to slurry was 0.8:1. The resultant mixture was divided into 6 samples, which were then sealed in separate polyethylene sample bottles and stored at room temperature for 1, 5, 12, 15, 19, and 22 days respectively.

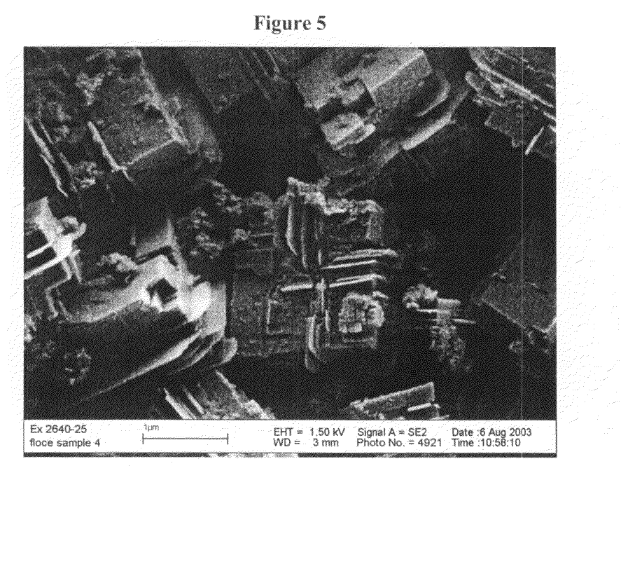

At the end of the storage time, each bottle was opened and the molecular sieves crystals were immediately separated from the slurry by washing, filtration and drying at 120° C. for 16 hours. The weight of the separated crystals was measured and the product yield (as a percentage of the total solids of the synthesis mixture) was plotted against storage time. The results are shown in FIG. 1, from which it will be seen that the yield loss was only 1.2 wt % even after 22 days storage. SEM of the product after 19 days storage is shown in FIG. 5 and showed no dissolution of the crystals compared to the starting material (FIG. 2).

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. A method of synthesizing a crystalline molecular sieve, the method comprising:
(a) forming a reaction mixture comprising a source of phosphorus, a source of aluminum, at least one organic directing agent and, optionally, a source of silicon;
(b) inducing crystallization of the crystalline molecular sieve from said reaction mixture to form a slurry, the slurry comprising said crystalline molecular sieve;
(c) maintaining said slurry in contact with a flocculant for a period of 12 hours to 30 days; and thereafter
(d) recovering said crystalline molecular sieve from said slurry.

2. The method of claim 1, wherein said slurry is maintained in contact with said flocculant for a period of 12 hours to 30 days.

3. The method of claim 1, wherein said slurry is maintained in contact with said flocculant for a period of 24 hours to 20 days.

4. The method of claim 1, wherein said slurry is maintained in contact with said flocculant for a period of 72 hours to 5 days.

5. The method of claim 1, wherein the amount flocculant present during (c) is between about 0.005% and about 0.100% by weight of the crystalline molecular sieve.

6. The method of claim 1, wherein the amount of flocculant present during (c) is between about 0.01% and about 0.05% by weight of the crystalline molecular sieve.

7. The method of claim 1 and including the further step of diluting the slurry with water so that the volume ratio of slurry to water diluent is between 1:0.5 and 1:1.5.

8. The method of claim 1 and including the further step of diluting the slurry with water so that the volume ratio of slurry to water diluent is between 1:0.7 and 1:1.2.

9. The method of claim 1, wherein said flocculant is an organic polymer.

10. The method of claim 1, wherein said flocculant is a polyethyleneimine.

11. The method of claim 1, wherein said crystalline molecular sieve is selected from a CHA framework-type molecular sieve, an AEI framework-type molecular sieve and an intergrowth of CHA and AEI framework-type molecular sieves.

12. The method of claim 1, wherein said crystalline molecular sieve comprises a silicoaluminophosphate and/or an aluminophosphate.

13. A method of increasing the storage life of an as-synthesized silicoaluminophosphate and/or an aluminophosphate molecular sieve comprising adding a flocculant to a slurry comprising crystals of said molecular sieve and a liquid medium used in the crystallization of said molecular sieve.

14. The method of claim 13, wherein said flocculant is an organic polymer.

15. The method of claim 13, wherein said flocculant is a polyethyleneimine.

16. A molecular sieve produced by the method of claim 1.

17. A process for conversion of an oxygenate-containing feedstock to a product comprising olefins comprising contacting said feedstock with a catalyst comprising the molecular sieve of claim 16.

18. The process of claim 17, wherein said feedstock contains methanol and/or dimethyl ether and said product comprises ethylene and propylene.
